(19) United States
(12) Patent Application Publication
(10) Pub. No.: US 2008/0132999 A1
(43) Pub. Date: Jun. 5, 2008

(54) Tubular Polymer Stent Coverings

(76) Inventors: Robert A. Mericle, Brentwood, TN (US); Swadeshmukul Santra, Orlando, FL (US); Christophe D. Batich, Gainesville, FL (US); Matthew V. Burry, Springfield, MO (US); Courtney S. Watkins, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/547,703

(22) PCT Filed: Jul. 11, 2005
(86) PCT No.: PCT/US05/24609

§ 371 Date: Jan. 31, 2008

Related U.S. Application Data
(60) Provisional application No. 60/586,552, filed on Jul. 9, 2004.

(51) Int. Cl. A61F 2/82

(52) U.S. Cl. 623/1.34; 623/1.46; 623/1.42

(57) ABSTRACT

The present invention concerns thin, flexible tubings useful as stent coverings; covered stents; methods for applying such coverings to stents; and methods for reinforcing biological lumens, such as the intracranial vasculature, by introducing the covered stent into the biological lumen and positioning the covered stent at a target site, such as the site of a vascular defect.

### CROSS-REFERENCE TO RELATED APPLICATION

The present application claims benefit of U.S. Provisional Application Ser. No. 60/586,552, filed Jul. 9, 2004, which is hereby incorporated by reference herein in its entirety, including any figures, tables, nucleic acid sequences, amino acid sequences, and drawings.

### BACKGROUND OF THE INVENTION

The present invention generally relates to intravascular stents and more particularly pertains to covers that envelop the stent's supporting structure.

Stents or expandable grafts are implanted in a variety of body lumens in an effort to maintain patency and/or to treat disease. These devices are typically intraluminally implanted by use of a catheter which is inserted at an easily accessible location and then advanced through the vasculature, for example, to the deployment site. The stent is initially maintained in a radially compressed or collapsed state to facilitate maneuvering within the body lumen. Once in position, the stent is deployed which, depending on its construction, is achieved either automatically (e.g., by the removal of a restraint) or actively (e.g., by the inflation of a balloon about which the stent is carried on the catheter).

Various stent configurations are well known and typically comprise a generally tubular arrangement of struts, spines, wires, etc. Such elements are advantageously positioned, oriented, and interlinked to enable the stent to be expanded and to then provide the required radial support to lumen walls in which they are deployed. The particular stent configuration that is employed along with the material or materials selected for its construction determine the performance characteristics of the resulting device. Such characteristics include, but are not limited to, radial stiffness, longitudinal flexibility, longitudinal stability upon expansion, expansion ratio, coverage area, etc. Most stent configurations that are presently known have a fairly open structure with a commensurately limited coverage area. As a stent is expanded, a given area of support structure becomes distributed over an even greater area. This is especially aggravated in the event the stent configuration is selected so as to maintain a constant length during expansion.

The voids between the various support elements of the stent can be problematic. For example, lumen wall tissue directly adjacent to such voids is not supported and can prolapse. Similarly, plaque or other material not directly supported by a stent element could also prolapse or come loose and be swept downstream to cause an embolism. As a result, it is usually most desirable for a stent to provide as much coverage as possible.

Another problem encountered with conventional stent configurations is the risk of over-expanding the stent so as to unnecessarily traumatize or otherwise distort the body lumen at the deployment site. While a particular stent configuration may inherently limit the maximum diameter that can be achieved, such stents can nonetheless exceed the maximum diameter that can be tolerated by the body lumen at the deployment site. Additionally, it may be desirable for certain portions of the stent to expand to a greater diameter than other portions of the stent. Conversely, it may be desirable for the stent to achieve a constant diameter over its entire length in contrast to the natural “bow-tie” effect many configurations are prone to. This occurs because a stent's resistance to expansion near its ends is often less than near its center and because of the expansion characteristics of inflatable balloons at the balloon tapers.

Stents are generally placed inside a blood vessel after balloon angioplasty to prevent restenosis, both extra- and intra-cranially. Stents are also used to assist coiling of wide-neck intra-cranial aneurysms. Most stents are made out of metal alloys and are made self-expandable or balloon-expandable.

When stents are placed across the orifice of an aneurysm or arteriovenous fistula, additional embolic agents, such as coils, are necessary to prevent blood flow into the aneurysm lumen or fistula, which otherwise occurs due to the spaces that exist between the struts, spires, wires, etc. Thus, it is desirable to cover these spaces in order to prevent deleterious blood flow across the stent. Unfortunately, previous attempts at covering stents have caused undesirable changes to their biomechanical properties, such as increased stiffness, increased constrained diameter, decreased expanded diameter, decreased flexibility, etc.

Intracranial aneurysms and intracranial carotid-cavernous fistulae (CCF) are vascular malformations that occur in the intracranial compartment. An aneurysm is an abnormal dilation of a portion of an artery due to a weakening in the vessel wall either congenitally or by disease. The two main types of aneurysms that occur are saccular (berry) and fusiform aneurysms. Saccular aneurysms have a neck and involve part of the circumference of the wall whereas fusiform aneurysms do not have a neck and encompass the entire wall. Intracranial aneurysms can rupture and hemorrhage into the brain causing stroke with severe disability or death. An intracranial carotid-cavernous fistula is an abnormal direct connection between the intracranial carotid artery and the adjacent intracranial cavernous sinus (vein). In this situation, the high-pressure arterial blood is shunted directly into the low-pressure venous system without transversing the lengthy capillary bed. A CCF can cause venous hypertension, which can lead to a massive swollen and injected eye (proptosis and chemosis), glaucoma, blindness, deformity, and reduced blood perfusion to the brain.

Treatment of intracranial aneurysms has traditionally been accomplished by open craniotomy and aneurysm clipping. These surgical procedures are invasive and involve a high risk (Giannotta, S. L. and Litofsky, N. S. J Neurosurg., 1995, 83(3):387-393; Johnston, S. C. et al. Stroke, 2001, 32(3):597-605). Many non-invasive endovascular procedures have been developed to treat lesions such as intracranial aneurysms, intracranial CCF, and other pathological diseases that require redirection or restriction of blood flow in the brain. Endovascular balloons have been used for occlusion of CCF and parent arteries for treatment of aneurysms (Laitinen, L. and Servo, A. J Neurosurg., 1978, 48(2):307-308; Debrun, G. Radiology, 1979, 130(1):141-147). The use of thrombogenic coils for intracranial aneurysms was first performed in 1989, although coils were previously used for carotid artery occlusion (Braun, I. F. et al. AJNR Am. J. Neuroradiol., 1985, 6(6):953-956). Detachable coils are currently used for some saccular aneurysms with narrow necks, as the coils are placed within the aneurysm lumen, while maintaining patency of the parent artery. Many wide-necked or fusiform aneurysms cannot be treated with coils alone because the coils cannot be contained in the aneurysmal sac (Ewald, C. H. et al. Acta. Neurochir. (Wien), 2000, 142(7):731-737). Bare stents to assist in coiling some wide-necked aneurysms were first described in 1998 (Mericle, R. A. et al. Neurosurgery, 1998, 43(5):1229-1234; Mericle, R. A. et al. J. Neurosurg., 1998, 89(1):142-145). However, bare stents will never be useful for treatment of some complex wide-necked aneurysms, fusiform aneurysms, or CCF, because the blood flow is free to pass between the stent struts and into the aneurysm lumen or fistula rent.

Covered microstents could be used as a stand-alone treatment for some types of intracranial aneurysms and CCFs because the covering would prevent blood flow into the lesion. A covered stent could be placed in the parent artery to bridge the abnormality in order to occlude blood flow into the aneurysm lumen or CCF rents. The use of covered stents in the proximal carotid and vertebral arteries has been reported (Geremia, G. et al. AJNR Am. J. Neuroradiol., 1997, 18(2):271-277; Schellhammer, F. et al. Invest. Radiol., 1999, 34(1):22-27; Fontaine, A. B. et al. Cardiovasc. Intervent. Radiol., 2001, 24(5):324-328; Fontaine, A. B. et al. J. Vasc. Interv. Radiol., 2001, 12(4):487-492; Najibi, S. et al. J. Surg. Res., 2002, 106(1):15-19; Marty, B. et al. J. Vasc. Interv. Radiol., 2002, 13(6):601-607). For intracranial use, the covered stent should be miniaturized to fit the smaller, more complex arteries in the brain. The covered microstent should also be highly flexible and maneuverable for smooth navigation because of the increased tortuosity in the intracranial arteries. This requires that the covering material be thin, flexible, and should not move relative to the stent during or after placement.

Clinical application of covered microstents would be dramatically improved if one understood and could predict how the mechanical properties of the covered stent would behave upon deployment. The proper numerical analysis could help in designing covered stents and provide design-driven selection of suitable materials.

### SUMMARY OF THE INVENTION

The present invention pertains to thin, flexible tubings useful as stent coverings. One embodiment of the present invention concerns ultra-thin (ranging from about 20 microns to about 200 microns in thickness), seamless, highly flexible and tear-resistant polymer tubings useful as stent coverings. In another embodiment, the stent covering is about 150 microns in thickness. An additional aspect of the invention relates to methods for applying coverings of the invention to stents. Another aspect of the invention concerns stents covered with stent coverings of the subject invention. An additional aspect of the invention provides methods for treating a biological lumen by introducing a covered stent of the invention into the lumen.

Appropriate modeling of covered stents will assist in designing suitable coverings, and help to reduce the failure rate of covered microstents. The present inventors have used the finite element method to determine the mechanical properties of the covered microstent and investigate the effects of the covering on the mechanical behavior of the covered microstent. Variations in the mechanical properties of the covered microstent such as deployment pressure, elastic recoil and longitudinal shortening due to change in thickness and material properties of the cover have been investigated. This work is also important for custom-designing covered microstents, such as adding cutout holes to save adjacent perforating arteries.

The covered stents of the invention are particularly suitable for the treatment of intra-cranial wide-necked and fusiform aneurysms and fistulas, which are currently difficult or impossible to treat either surgically or endovascularly. The coverings may be custom-made in a variety of sizes and thickness and can be mounted easily over a variety of stents, including metallic stents, by a swelling procedure.

Optionally, the stent coverings of the present invention incorporate one or more types of contrast agent for in vivo imaging purposes. For example, the stent covering may incorporate one or more paramagnetic materials, such as paramagnetic iron oxide, dysprosium (Dys) oxide, and gadolinium (Gd) oxide, to make the stent covering detectable by magnetic resonance imaging (MRI). Optionally, the stent covering incorporates one or more radio-opaque agents, such as gold particles, to render the stent covering detectable by CT scan. The type of contrast agent will vary with the imaging modality to be utilized. One or more types of contrast agents can be used with the same stent covering, for multi-modal imaging. For example, multi-modal quantum dots may be utilized.

### BRIEF DESCRIPTION OF THE DRAWINGS

FIGS. 1A-1C are schematic diagrams of an “S” hook (FIG. 1A) and three point bend fixture (FIGS. 1B and 1C) that can be utilized for in vitro performance testing of the covered stents of the invention.

FIGS. 2A-2D are schematic diagrams of the in vitro flow system that can be utilized for in vitro performance testing of the covered stents of the invention.

FIG. 3 shows an angiogram demonstrating blood flow through a previously created arteriovenous fistula (AVF) in a New Zealand White rabbit common carotid artery.

FIG. 4 shows an angiogram of the common carotid artery immediately after placement of a covered stent of the invention across the AVF. The AVF is no longer receiving blood flow.

FIG. 5 shows a follow-up angiogram of the AVF treated with the covered stent in FIG. 4. Normal antegrade carotid blood flow remains.

FIG. 6 shows a diagram of an intracranial artery segment with a fusiform aneurysm.

FIG. 7 shows a photograph of a Palmaz-Schatz balloon expandable microstent in its constricted form.

FIG. 8 shows a nominal stress versus strain curve for SILASTIC T2 silicone (10 samples).

FIG. 9 shows a mesh scheme of the covered microstent.

FIGS. 10A and 10B show a free expanded microstent at 3-mm-diameter with a diamond shape slot, as used in an in vitro experiment (FIG. 10A); and as depicted by finite element (FE) results.

FIG. 11 shows deformation of a covered microstent.

FIG. 12 shows a graph of deployment pressure as a function of the central outer diameter of a bare stent.

FIGS. 13A and 13B show the deployment pressure required to reach 1.57-mm-diameter in the central area of the stent (P_{1.57 mm}) for cases with different covering thickness t, different Young's modulus of the covering E_cover (FIG. 13A); and P_{1.57 mm} & P_{1.54 mm} versus covering thickness (FIG. 13B).

FIGS. 14A and 14B show longitudinal shortening at different deformation diameters for different covering thickness (FIG. 14A), and elastic recoil for different covering thickness.

FIG. 15 is a photograph showing a 3-point bend test apparatus depicted in FIGS. 1A-1C.

FIG. 16 is a graph of force vs. displacement for each bare and covered stent.

FIG. 17 is a graph of load vs. displacement for covered silicone rods. Each trace is for one representative sample.

FIG. 18 is a bar graph showing force measured at 1 and 3 mm for covered silicone rods.

FIG. 19 is a graph of load vs. displacement for bare stent and elastomeric coverings. Data for coverings was calculated by subtracting the average curve for silicone rod from curves for representative covered silicone rod samples.

### DETAILED DESCRIPTION OF THE INVENTION

The polymer tubings or stent coverings of the present invention may be constructed from a variety of biocompatible thermoplastic elastomers, including polyurethanes (e.g., TECOFLEX), silicone-polyurethane copolymer (e.g., CARBOSIL, PURSIL), styrene-ethylene-butylene-styrene (C-FLEX), and silicone. The sized polymer tubing may be swelled in an appropriate solvent (up to 300% surface area) and mounted on the stent by placing it over the stent, thereby making the device a covered stent. The covered stent will then be allowed to dry in an appropriate drying chamber. Because of the properties of the material(s) of construction, the covered stent will advantageously remain as flexible as the uncovered (e.g., naked metallic) stent, with the stent covering exerting minimal recoil pressure on the outer surface of the stent.

Any biocompatible, medical grade elastomer would be suitable for construction of the polymer stent coverings of the invention. For example, the polymer stent coverings of the present invention may be constructed of any of the elastomers disclosed in U.S. patent publication no. 20050049691 (“Polymeric reconstrainable, repositionable, detachable, percutaneous endovascular stentgraft”; Mericle et al.; filed Sep. 2, 2003) as useful for the construction of the double lumen tubes described therein. U.S. patent publication no. 20050049691 is incorporated herein by reference in its entirety. In one embodiment, the stent covering of the invention is a polymer tubing, wherein the polymer may be swelled using an appropriate swelling agent, such as a solvent. Appropriate swelling agents are capable of swelling the polymer without dissolving it. For example, for CARBOSIL 40 90A (The Polymer Technology Group Inc., Berkeley, Calif.), which is a solution grade elastomeric, tear-resistant silicone-polyurethane thermoplastic copolymer, a swelling solvent of 70% acetone in water (V/V) for 5 minutes is suitable. For C-FLEX tubing material, which is a styrene-ethylene-butylene-styrene block copolymer with excellent tensile and tear strength (CONSOLIDATED POLYMER TECHNOLOGIES, Inc., Clearwater, Fla.), a swelling solvent of n-hexane was suitable. Another solvent that may be used as a swelling agent is acetone. Mixed solvents, such as acetone/water mixtures, can be used to swell the polymer stent covering without dissolving it.

Thermoplastic elastomers (randomly entangled polymer chains) can be swelled (when solvent molecules penetrate) and also can be dissolved (polymer chains are no longer entangled in this situation) if appropriate solvent is selected. Thermoplastic elastomers are cross-linked polymers (individual polymer chains are linked through bonding); they can be swelled but cannot be dissolved without breaking those crosslinks.

The swellable stent coverings may be lubricated or non-lubricated. In another embodiment, the stent covering of the invention is a polymer tubing that is lubricated. The lubricated stent covering may be swellable or non-swellable.

The dimensions of the stent coverings of the present invention depend upon the dimensions of the underlying stent, the size of the biological lumen to be reinforced (e.g., intracranial vessel), and the nature of the luminal defect (e.g., vascular defect). Examples of stent lengths include, but are not limited to, 10 mm, 15 mm, 20 mm, 25 mm, 30 mm, and 35 mm. Examples of stent diameters include, but are not limited to, 3 mm, 3.25 mm, 3.5 mm, 3.75 mm, 4 mm, 4.25 mm, 4.5 mm, 4.75 mm, and 5 mm. Examples of stents, including radially expandable stents, that may be covered with the stent coverings of the present invention, stent materials, and stent dimensions are described in U.S. Pat. Nos. 6,214,039; 4,733,665; 4,739,762; 5,102,417; 4,580,568; 4,907,336; 4,740,207; 4,886,062; 4,969,458; 5,163,958; 5,195,984; 5,514,154; and 6,488,701; each of which are incorporated herein by reference in their entirety.

Optionally, stent coverings of the subject invention having the desired dimensions of length, thickness, and inner diameter may be made to occupy a rolled configuration (resembling a condom), which can then be easily placed over the stent by rolling the covering over the stent. No swelling of the covering is necessary.

The stent coverings of the invention may be custom made to fit any stent, and the thickness of the cover may also be adjusted; however, an ultra thin covering having a thickness within the range of about 20 microns to about 200 microns is preferred. The covered stent can be easily maneuvered via catheter through the patient's vascular system to the site of the vascular defect. The endovascular treatment of difficult intra-cranial aneurysms and fistulas will thus be possible. For aneurysms with wide necks, the covered stent will be placed at the neck of the aneurysm, preventing blood from filling the aneurysm sac, and thereby decreasing the risk of aneurysm rupture and resultant intra-cranial hemorrhage. For fusiform aneurysms, the stent may be placed within the body of the aneurysm, restoring natural vascular anatomy and blood flow, thereby decreasing the risk of aneurysm growth, rupture, and resultant neurological decline. For arteriovenous fistulas, the stent may be used to line the injured vessel, restoring natural vascular anatomy and blood flow while allowing the vascular defect to heal.

The present invention includes a method for treating a biological lumen (such as a blood vessel having a vascular defect) by introducing a covered stent of the invention at a target site within the lumen. Treatment with the covered stents of the present invention will simplify the treatment process, minimize risk, improve patient outcome, and decrease overall treatment cost. Because of location and morphology, many intra-cranial aneurysms are untreatable or difficult to treat with current surgical and endovascular methods and devices. In addition, surgical treatment is invasive, requiring craniotomy with resultant lengthy hospital stay, recovery period, increased discomfort to the patient, and increased risk of infection. At this time, endovascular treatment of fusiform aneurysms is extremely difficult and often impossible, and endovascular treatment of intra-cranial arteriovenous fistulas requires the complete and permanent occlusion of the afflicted vessel. Minimally invasive endovascular treatment of these conditions with a covered stent of the present invention will preserve or restore natural vascular anatomy and blood flow. In the case of aneurysms, treatment with a covered stent of the invention will decrease the risk of aneurysm rupture and resultant intra-cranial hemorrhage. With arteriovenous fistulas, such treatment will preserve blood flow through the damaged vessel and promote healing of the defect.

Optionally, the covered stents of the subject invention can be used in conjunction with various pharmacologic substances, such as substances that breakup or dissolve vascular occlusions, and/or prevent the formation of vascular occlusions. For example, in the case of blood clots (also known as thrombi), various anticoagulant, thrombolytic (so called “clot-busting” drugs) or anti-platelet agents can be administered orally or intravenously. Examples include heparin (CALCIPARINE, HEPATHROM, LIP-HEPIN, LIQUAEMIN, PANHEPRIN), warfarin (ATHROBMIN-K, PANWARFIN), tissue plasminogen activator (tPA; ALTEPLASE; ACTIVASE), streptokinase (KABIKINASE, STREPTASE), urokinase (ABBOKINASE), anistreplace, aminocaproic acid, aprotinin, acetylsalicylic acid (aspirin), dipyridamole (PERSANTINE), abciximab (REOPRO), dalteparin (FRAGMIN), enoxaparin (LOVENOX), hirudin (DESIRUDIN), 4-hydroxycoumarin (COUMADIN), lepirudin (REFLUDAN), protamine sulfate, phytonadione (Vitamin K₁), reteplase (RETAVASE), clopidogrel (PLAVIX), and ticlopidine (TICLID). Many of these agents operate by inhibiting the clotting mechanism (anticoagulants), lysing thrombi (fibrinolytic agents), and interfering with platelet adhesion and/or aggregation.

The stent or stent covering can be impregnated or coated with one or more biologically active agents, such as pharmacologic substances that breakup or dissolve vascular occlusions. The biologically active agents can function on contact with the covered stent or the substances can be released from the covered stent into the vasculature in a controlled release fashion. Optionally, the biologically active agents can be released and/or become activated upon contact with blood, or otherwise be responsive to the physiological environment. For example, the pharmacologic substance can be coated on the interior wall of the stent, and thereby exposed to blood passing through the stent, or the pharmacologic substance can be impregnated or coated on the stent covering, and thereby exposed to the vascular tissue of the lumen. The biologically active agents can be temperature-sensitive and/or pH-sensitive, for example. As an alternative to impregnated or coated components, the biologically active agent can be delivered by other means, such as a port on the stent that permits injection of the biologically active agent at a target site. As used herein, the term “biologically active agent” refers to any substance that is capable of promoting or causing a therapeutic effect in a patient.

Methods known in the art for insertion/introduction and operation of a stent can also be utilized with the covered stent of the present invention. For example, the covered stent of the present invention can be introduced into a blood vessel through an introducer (also known as an introducing catheter), which is used to access the vessel. Typically, guide wires will be utilized.

Optionally, any component stent covering or covered stent of the subject invention can be at least partially composed of an imageable material. For example, the covering or stent can be composed of an imageable material. As used herein, an “imageable material” includes those materials the location of which can be discerned within a given opaque, ambient medium such as biological tissue, using the appropriate sensing equipment, such as imaging equipment. The imageable material selected should have an image “signature” discernibly different from that of the surrounding medium into which the covered stent is to be introduced. Components of the covered stent (e.g., the stent and/or stent covering) can be coated or impregnated with one or more imageable materials, for example.

In one embodiment, the imageable material is an echogenic material with an acoustic impedance different from that of the surrounding medium (i.e., high acoustic impedance differential), enabling the covered stent to be imaged using a sonic imaging device (e.g., ultrasound imaging equipment). A variety of materials that are echogenic (i.e., sound reflective) can be utilized, such as aluminum, hard plastic, sand, and metal particles. For example, the echogenic material can be any of those materials described in U.S. Pat. No. 5,201,314 and U.S. Pat. No. 6,106,473, or a combination of those materials. In another embodiment, the imageable material is a radio-opaque material (such as barium sulfate and/or tantalum) that can be imaged with radiographic equipment (e.g., an x-ray machine or computed tomography (CT) scanner). In a further embodiment, the imageable material is a substance that can be imaged using magnetic resonance imaging/spectroscopy (MRI/MRS) equipment, such as gadolinium (Gd). Other imageable materials include those materials detectable through single photon emission tomography (SPECT) or positron emission tomography (PET), for example. The component or components of the covered stent can be wholly or partly composed of the imageable material. As indicated above, the imageable material can be in the form of a coating or film on an underlying substrate.

Contrast media, such as dyes, can also be used in conjunction with the appropriate imaging equipment in order to discern more details in vivo within the biological lumen (such as the blood vessel). For example, barium-containing and iodine-containing dyes can be administered in conjunction with x-ray or CT imaging.

The stent covering may be coated with or otherwise incorporate one or more paramagnetic materials, such as paramagnetic iron oxide, dysprosium (Dys) oxide, and Gd oxide, to make the stent covering detectable in vivo by magnetic resonance imaging MRI). Optionally, the stent covering incorporates one or more radio-opaque agents, such as gold particles, to render the stent covering detectable by CT scan. The type of contrast agent will vary with the imaging modality to be utilized. One or more types of contrast agents can be used with the same stent covering, for multi-modal imaging. For example, the coverings may be coated or otherwise incorporate nanoparticles such as multi-modal quantum dots. See, for example, Santra S. et al., Chem. Commun. (Camb), 2005, Aug. 7, 25:3144-3166 (Epub 2005, May 19); Santra S. et al., J. Am. Chem. Soc., 2005, 127(6):1656-1657; Santra S. et al., Chem. Commun. (Camb)., 2004, Dec. 21, 24:2810-2811 (Epub 2004, Oct. 25); Santra S. et al., J Nanosci. Nanotechnol., 2004, 4(6):590-599; each of which are incorporated herein in their entirety.

In one embodiment, the covered stent is used to treat intracranial aneurysms or intracranial carotid-cavernous fistulae (CCFs). For example, the stent can be placed in the parent artery to bridge the vascular abnormality to occlude blood flow into the aneurysm lumen or CCF rents (e.g., rents in the walls of the intracavernous carotid artery or its branches). In addition to blood vessels (veins, arteries, etc.), the covered stents of the invention can be placed in other biological conduits, such as a ureter, urethra, fallopian tube, bile duct, intestine, and the like.

The covered stent of the present invention can be placed in biological lumens within humans or animals, such as non-human mammals. Thus, the covered stent of the present invention can be used in a variety of veterinary applications in order to treat domesticated or non-domesticated animals. The dimensions of the stent and stent covering can be optimized for the particular animal subject.

As used herein, the term “stent” includes any device that is introduced intravascularly in an effort to prevent restenosis. Stents are typically short flexible cylinders or scaffolds, made of metal or polymers, and are often placed into a vessel to maintain or improve blood flow. Various types of these devices are widely used for reinforcing diseased blood vessels, for opening occluded blood vessels, and for defining an internal lumen to relieve pressure in an aneurysm. Stents allow blood to flow through the vessels at an improved rate while providing the desired lumen opening or structural integrity lost by the damaged vessels. Some stents are expanded to their proper size by inflating a balloon catheter, referred to as “balloon expandable” stents, while others are designed to elastically resist compression in a “self-expanding” manner.

Balloon expandable stents and self-expanding stents are generally delivered in a cylindrical form, compressed to a smaller diameter and are placed within a vessel using a catheter-based delivery system. When positioned at a desired site within a vessel, these devices are expanded by a balloon, or allowed to “self-expand” to the desired diameter.

While certain types of stents such as braided metal stents may be preferred for some applications, the stent covering, covered stent, and methods of the present invention are not so limited and can be used on a wide variety of prosthetic devices. Thus, in the case of stents, the present invention also applies, for example, to the class of stents that are not self-expanding, including those which can be expanded, for instance, with a balloon; as well as polymeric stents of all kinds. Other medical devices that can benefit from the coverings of the present invention include blood exchanging devices, vascular access ports, central venus catheters, cardiovascular catheters, extracorpeal circuits, vascular grafts, pumps, heart valves, and cardiovascular sutures, to name a few. Regardless of detailed embodiments, applicability of the invention should not be considered limited with respect to implant design, implant location or materials of construction. Further, the present invention may be used with other types of implantable prostheses.

All patents, patent applications, provisional applications, and publications referred to or cited herein, whether supra or infra, are incorporated herein by reference in their entirety to the extent they are not inconsistent with the explicit teachings of this specification.

Following are examples which illustrate procedures for practicing the subject invention. These examples should not be construed as limiting. All percentages are by weight and all solvent mixture proportions are by volume unless otherwise noted.

### Example 1

### Evaluation of Materials for Stent Coverings

Tear-resistant, biocompatible elastomeric polymer materials will be chosen for making tubes. Two types of commercially available solution grade (SG) thermoplastic materials will be selected: a polyurethane (TECOFLEX SG-80A, THERMEDICS Polymer Products, Woburn, Mass.) and a few silicone-polyurethane copolymers (CARBOSIL 40 90A, PURSILI 20 80A and PURSIL AL5 75A, The POLYMER TECHNOLOGY GROUP Inc., Berkeley, Calif.). TECOFLEX SG-80A is an aliphatic polyether based thermoplastic polyurethane (TPU). PURSIL silicone-polyether-urethane and CARBOSIL silicone-polycarbonate-urethane are thermoplastic copolymers containing silicone in the soft segment. PURSIL 20 80A is an aromatic silicone polyetherurethane whereas PURSIL AL5 75A is an aliphatic silicone polyetherurethane. Table 1 lists some of the physical test data of these materials reported by the manufacturers. From the table it is clear that these materials cover a range of mechanical properties. In studying all four materials, the purpose is to find the right material for the optimized device.

TABLE 1

| | Tensile | Tensile | | | |
|---|---|---|---|---|---|
| | Stress at | Stress at | Ultimate | | Tear |
| | 100% | 300% | Tensile | Ultimate | Strength, |
| | Elongation | Elongation | Strength | Elongation | die |
| Elastomer | (psi) | (psi) | (psi) | (%) | “C” (pli) |
| TECOFLEX | 300 | 800 | 5800 | 660 | N/A |
| EG-80A* | | | | | |
| PURSIL | 270 | 570 | 5300 | 900 | 390 |
| 20 80A | | | | | |
| PURSIL | 900 | 1630 | 4900 | 770 | 115 |
| AL5 75A | | | | | |
| CARBOSIL | 1310 | 2400 | 4300 | 530 | 500 |
| 40 90A | | | | | |

*Represents the test data for extrusion grade. The solution grades differ from the extrusion grades in that they contain no melt processing lubricants.

If necessary, in order to clearly visualize the covered stent under a fluoroscope while performing in vivo tests, the tubes can be made radio-opaque. Radio-opaque grade TECOFLEX is available with 20 wt % and 40 wt % loading of barium sulfate. Other polymers could be either blended with barium sulfate or could be custom-blended from the manufacturer.

Thermoplastic C-FLEX medical grade tubing will be selected for the present experiment. A double lumen stent will be custom made by MEDICAL EXTRUSION TECHNOLOGIES, Inc., Murrieta, Calif. C-FLEX tubing material is a styrene-ethylene-butylene-styrene block copolymer with excellent tensile and tear strength (CONSOLIDATED POLYMER TECHNOLOGIES, Inc., Clearwater, Fla.). Custom tubing formulations with radio-opaque grade are available. C-FLEX medical grade tubing complies with USP 24/NF19, Class VI testing. They are non-pyrogenic, non-cytotoxic, compatible with tissue, non-hemolytic with low platelet adhesion and low protein binding. Current medical applications of the material include medical fluid/drug delivery, dialysis, and cardiac by-pass. C-FLEX tubing is significantly less permeable than silicone and they are heat sealable and bondable. Tubes of 150 μm, 200 μm, 250 μm and 300 μm thicknesses will be made to determine the optimal thickness of the graft. The manufacturer will have the capability to make the tubes with a minimum of 150 μm thickness by the extrusion method. For intracranial applications these selected tube thicknesses are quite feasible.

### Example 2

### In Vitro Performance Testing of Covered Stent

Tests will be performed to evaluate the robustness of the device fabrication and to determine the device performance in vitro under various conditions.

A. Mechanical Testing of the Covered Stent by INSTRON

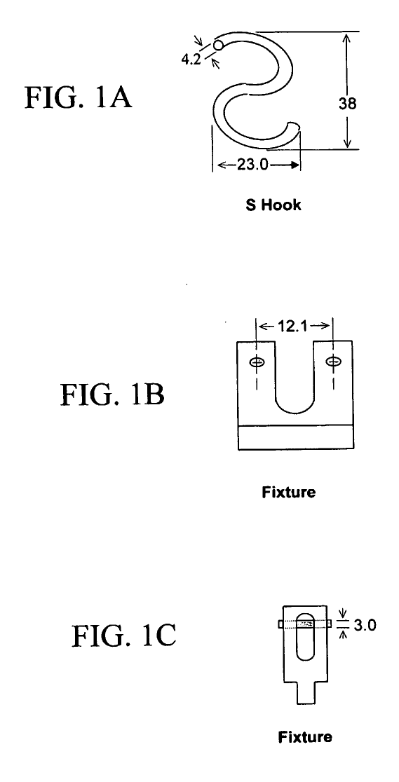

Flexibility and maneuverability testing is useful to predict the feasibility of navigating the device through the tortuous intracranial vasculature system in the brain before the final deployment. A three-point bend test on the double lumen tubular micrograft with uncured adhesive will be performed. FIGS. 1A-1C show an “S” hook (FIG. 1A) and three-point bend fixture (FIGS. 1B and 1C). The bend test fixture and an “S” hook for the bend test will be made in the machine shop.

Mechanical testing on the deployed covered stent will be performed using an INSTRON model 4301 (INSTRON Corporation, Canton, Mass.). Compression and three-point bend tests will be performed. The compression test will evaluate how much radial pressure (intra-arterial pressure) the covered stent can tolerate before it deforms. The three-point bend test will help to evaluate the flexibility of the deployed covered stent.

B. Performance Testing of the Device in an In Vitro Flow System

An in vitro flow system will be used for device performance testing. The flow system will approximate the blood circulation in the brain. Simulated blood fluid (SBF) will be circulated through the flow system in a pulsatile manner. Three models will be made: a human middle cerebral artery model (MCA) for flexibility and maneuverability testing, an aneurysm model, and an AVF model for the device in vitro performance testing. The feedback from in vitro testing will help further the development of the design and fabrication process. A repeated redesigning and remanufacturing process will be carried out to optimize the device performance.

The following materials will be used in the in vitro flow system: MASTERFLEX variable speed peristaltic pump (Cole Parmer, Niles, Ill.), TYGON tubing (FISHER SCIENTIFIC, Fairlawn, N.J.), quick disconnect fittings (FISHER SCIENTIFIC, Norcross, Ga.), polyethylene and polypropylene tubings (Clay Adams, Parsippany, N.J.), sheath introducer (CORDIS Endovascular Systems, Miami, Fla.), a 2-way PTFE plug stopcock (KIMAX, Kimble-Kontes, Vineland, N.J.) as flow resistor, a SHIMPO digital force gauge, model FGV (A)-5A, capacity 5.0 lb (DAVIS INOTEK Instruments, Baltimore, Md.) and a closed reservoir. The SBF will be used for flow experiments. The SBF will be composed of the following materials: poly(vinyl alcohol) (PVA) with a molecular weight of 93,400 (EASTMAN KODAK, Rochester, N.Y.), sodium chloride (NaCl) (FISHER SCIENTIFIC, Fairlawn, N.J.), boric acid (SIGMA Chemicals, St. Louis, Mo.), and sodium tetraborate decahydrate (ALDRICH Chemical Company, Inc., Milwaukee, Wis.).

The following materials and equipment will be used for the data acquisition component of the flow system: a desktop computer, a multifunction I/O data acquisition board (Model PC-LPM-16/PnP) (NATIONAL INSTRUMENTS, Austin, Tex.), NI-DAQ software Version 6.7 (NATIONAL INSTRUMENTS, Austin, Tex.), LABVIEW 5.1 software (NATIONAL INSTRUMENTS, Austin, Tex.), an Archer breadboard (RADIO SHACK, Fort Worth, Tex.), a 50-pin ribbon cable, silicon pressure sensors with a range of 0 to 7.3 psi (MPX5050 series, MOTOROLA, Phoenix, Ariz.), and a flow sensor with a range of 60 mL/min to 1,000 mL/min (Model 101T, MCMILLAN Company, Georgetown, Tex.). The SBF was made using a procedure from Jungreis, C. A. and Kerber, C. W. AJNR Am. J. Neuroradiol., 1991, 12(2):329-330. First, 12.1 g of PVA will be dissolved in one liter deionized water. In a separate container, 23.2 g of sodium borate will be dissolved in deionized water. The two solutions will be mixed and diluted to three liters. Boric acid will then be added to lower the pH to 7.5.

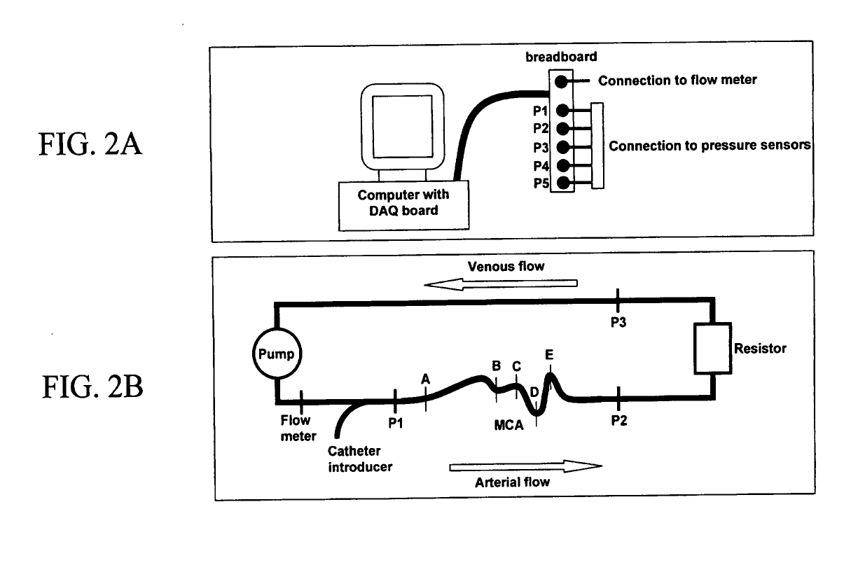

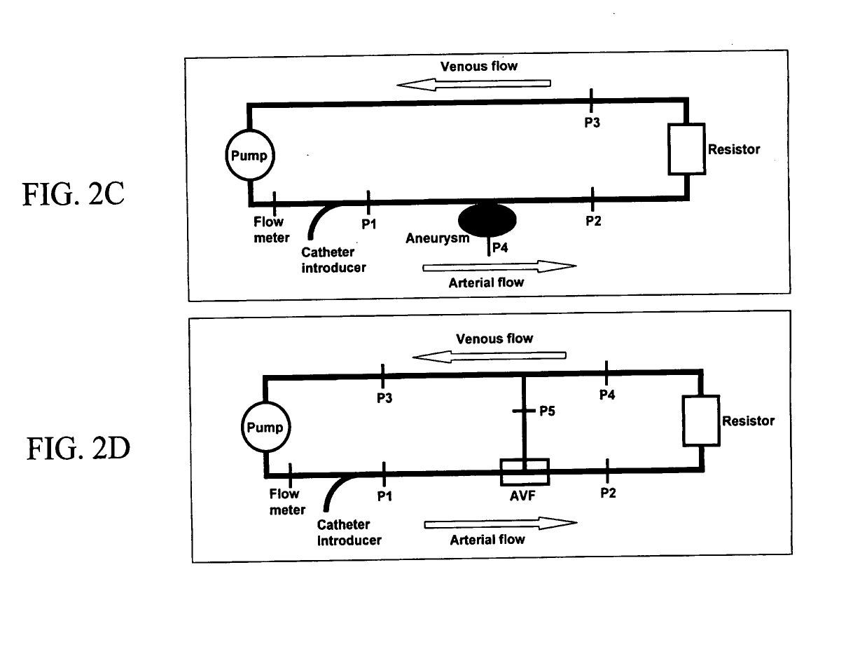

FIGS. 2A-2D show schematics of the in vitro flow system. The flow system includes two components: an electronic component and a flow component. The electronic component includes a breadboard and a computer with DAQ board. The computer is connected to the breadboard as shown in FIG. 2A. The flow component includes a peristaltic pump, a catheter introducer, a model of interest, a resistor, a flow meter, a closed reservoir (not shown in the schematic drawing) and several pressure sensors. They are all connected in a series. The performance of the covered stent will be studied in three different models: a MCA model (FIG. 2B), an aneurysm model (FIG. 2C) and an arteriovenous fistula model (AVF; FIG. 2C). The pressure sensors (P1, P2, P3, P4, and P5) will monitor the SBF pressure at different locations (as shown in FIGS. 2B, 2C, and 2D) and the flow meter will monitor the SBF flow in the flow system. All sensors (pressure and flow) will be connected to the breadboard. The resistor will model normal brain capillary bed. The micrograft device will be introduced through the catheter introducer port. The pulsatile flow rate will be controlled by the peristaltic pump.

C. Middle Cerebral Artery (MCA) Model for the Flexibility and Maneuverability Testing

A polypropylene tube of 2.5 mm interior diameter (ID) will be used for making the model of the tortuous MCA. This type of tube may kink while it is being bent to give the tortuous shape. To overcome this problem, an appropriate size copper wire will be inserted first inside the tube to give the desired shape. Translucent silicone adhesive (Silastic T2 from DOW CORNING) will be applied over the tube and then it will be heat treated. Once cured, silicone oil will be injected into the tube to facilitate removal of the support wire. The structure will be straightened and the copper wire support will be removed. Once released, the tube will return to its tortuous shape. The silicone over coating will reinforce and retain the structure. It will then be cleaned. The interior of the tube will be coated with a hydrophilic coating (HYDROMED C, CT BIOMATERIALS, CARDIOTECH International, Inc., Woburn, Mass.) and installed as shown in FIG. 2B. The MCA model will be connected to the flow system to test the flexibility and maneuverability of the micrograft device at different locations (A, B, C, D and E) as shown in FIG. 2B. The force required to push the catheter through the MCA model will be measured by a digital force gauge and then a histogram showing force at different locations will be created.

D. Aneurysm and AVF Model

As shown schematically, both aneurysm (FIG. 2C) and AVF (FIG. 2D) models will be made from silicone polyurethane copolymers (e.g., Carbosil 40 90A) by using appropriate molds. As described previously, the dip-coating method will be used to make tubular components for AVF and tubular and balloon components for aneurysm model. The interior of the balloon and the tubes will be coated with a hydrophilic coating (HYDROMED C, CT BIOMATERIALS, CARDIOTECH International, Inc., Woburn, Mass.). For in vitro performance testing, these models will be connected to the flow system.

### Example 3

### In Vivo Testing of Covered Stent in Rabbit Arteriovenous Fistula (AVF) Model

Under protocols approved by University of Florida's Institutional Animal Care and Use Committee, covered stents of the invention were tested for efficacy and histological compatibility in New Zealand White (NZW) rabbits.

Histological analysis of the effect of device placement was conducted in the normal common carotid artery. A vascular sheath was placed in the femoral artery of a NZW rabbit. Using endovascular techniques, a microstent covered with tubular polymer was navigated through the vasculature towards the common carotid artery. The device was deployed within the vessel and angiography was performed to confirm patency. Following device placement, the animal was monitored for a period of two to six weeks, depending on the experimental group to which it was assigned. At the end of the determined monitoring period, angiography of the stented vessel was performed to reveal angiographic patency. The animals were then euthanized and the vessels harvested for histological examination.

Efficacy was determined using a NZW rabbit arteriovenous fistula (AVF) model. An end-to-side AVF was created using the external jugular vein and common carotid artery. The next procedure was conducted after a healing period of about two weeks. Endovascular access was obtained through a vascular sheath in the femoral artery. A microstent covered with tubular polymer was advanced toward the AVF and placed across the lesion under fluoroscopic guidance. Once proper placement was determined, the covered microstent was deployed, occluding the AVF from arterial blood flow. Angiography was used to determine successful occlusion of the lesion from flow. The animals were monitored for periods of three to six weeks. Endovascular access was again obtained and angiography was performed in the same manner to evaluate the result of AVF treatment with the device. Following the evaluative angiography, the animal was euthanized and the vessels and device were removed for histology.

Various tubular polymeric materials were compared in these in vivo trials. Preliminary results are encouraging. The refined device was easily and reliably deployed. Placement of microstents covered with tubular polymers in normal carotid arteries resulted in minimal neointimal proliferation. In the AVF model, the device successfully occluded the lesion from flow while restoring normal flow through the primary vessel. After the monitoring periods, normal flow was still present and the lesions eliminated from circulation.

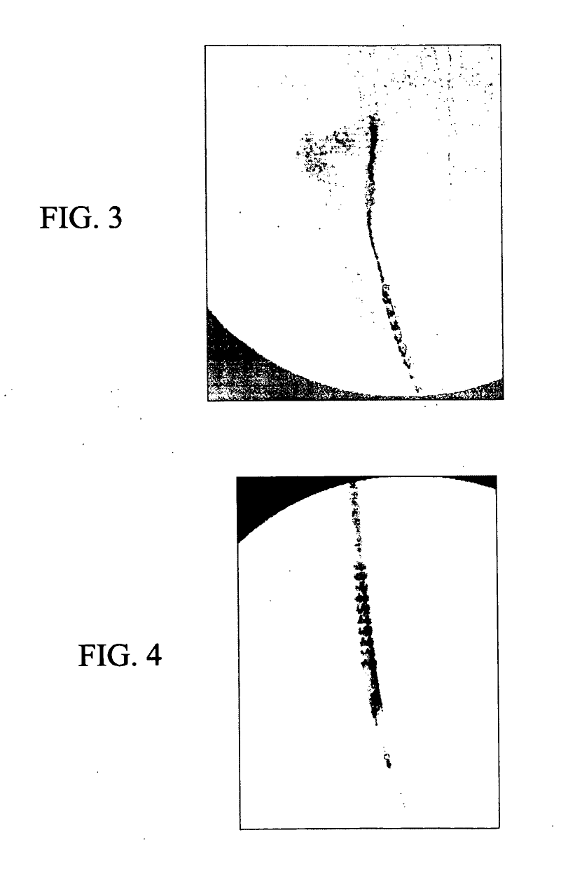

FIG. 3 shows an angiograph demonstrating blood flow through a previously created arteriovenous fistula in a New Zealand White rabbit common carotid artery. The normal antegrade carotid flow is diverted into the large jugular vein. No appreciable blood flow is going beyond the AVF. This lesion is to be treated by the microstent covered with tubular polymer. The undeployed microstent is seen at the bottom FIG. 3.

FIG. 4 shows an angiograph of the common carotid artery immediately after the covered microstent was placed across the AVF. As shown in FIG. 4, the AVF is no longer receiving blood flow. A normal antegrade flow pattern through the common carotid is restored. The microstent covered with tubular polymer, styrene-ethylene-butylene-styrene (C-FLEX) has occluded all blood flow from the fistula.

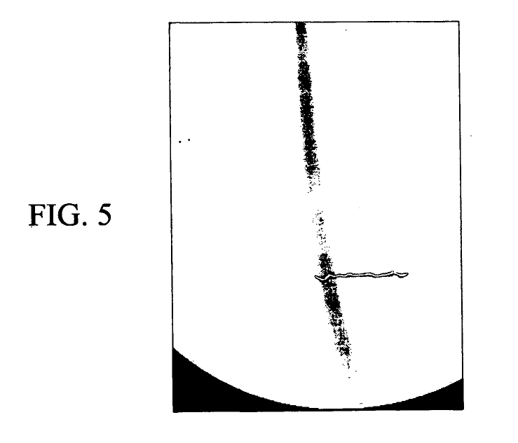

FIG. 5 shows a follow-up angiograph of the AVF treated with the microstent covered with tubular polymer (styrene-ethylene-butylene-styrene, C-FLEX). Normal antegrade carotid blood flow remains. The created AVF remains excluded from receiving blood flow. Also note that the lumen of the carotid does not show any abnormality from the microstent covered with tubular polymer.

### Example 4

### In Vivo Testing of Covered Stent in Normal Common Carotid Artery (CCA)

In these experiments, an ultra thin silicone covering of appropriate size was mounted onto a small Palmaz-Schatz stent before deployment. The effects of the covering on the primary mechanical performance of the stent (deployment pressure, elastic recoil and longitudinal shortening) were investigated by using finite element (FE) method. The results were compared to the mechanical properties of the microstent without covering. This is necessary to determine optimal deployment pressures, elastic recoil and longitudinal shortening for each desired diameter of artery where the covered stent would be placed in an intracranial artery.

Finite element analysis has been used to study mechanical properties of metallic stents, such as deployment pressure, elastic recoil and flexibility. Several studies have addressed the effects of stent geometries, the interaction between the stent and catheter balloon, or interactions between the stent and arterial wall. Auricchio et al. (2000) studied the biomechanical interaction between a balloon-expandable stent and a stenotic artery. Etavea et al. (2001) determined the exact mechanical characteristics of two different types of stents: tubular stents and coil stents. Migliavacca, F. et al. (J. Biomech., 2002, 35(6):803-811) investigated the effects of different geometrical parameters of a typical diamond-shaped coronary stent on the mechanical performance, and gave some suggestions for optimizing stent shape and performance. Rogers et al. (1997) studied a 2D balloon-artery interaction. However, no numerical analysis of covered stents has been published. The present inventors have used the finite element method to predict the mechanical properties of covered microstents which is a critical step towards using these covered microstents to treat many difficult aneurysms and CCF.

The effect of covered stent placement was initially evaluated by histological analysis of the normal common carotid artery (CCA) in the New Zealand White (NZW) rabbit model. In a typical procedure, a vascular sheath was placed in the femoral artery of a NZW rabbit. Using endovascular techniques, a silicone-covered balloon-expandable stent device was navigated through the vasculature towards the CCA. The device was deployed within the vessel and angiography was performed to confirm patency. Following the device placement, the animal was monitored for six weeks. Angiography of the stented vessel was then performed to reveal angiographic patency. The animals were then euthanized and the vessels harvested for histological examination. Under this preliminary investigation, six animals were selected. In three cases, the covering ripped during stent deployment (at approximately 4 atm pressure) and stenosis occurred in one case. For the other cases, the placement of these covered microstents in the rabbit common carotid artery (CCA) resulted in minimal neointimal proliferation, which demonstrated the feasibility of using covered stents. Moreover, the covered stent device was easily navigated through the vasculature system, which demonstrates flexibility and maneuverability of this covered microstent.

In this animal study, approximately 150 μm thick SILASTIC T2 silicone tubing elastomerically captured on a balloon expandable metal stent was used. Although the stent is well optimized for clinical applications, there will be variations in the mechanical properties such as deployment pressure, elastic recoil etc. when it is covered with elastomeric covering. In general, irrespective of the type of metal stent, the alteration in mechanical properties of a covered stent could be directly correlated to the mechanical properties of coverings. For intracranial applications, it is preferred to select an ultra-thin covering in such a way that the covering will have minimal impact on the flexibility and maneuverability of the balloon-expandable stent system. However, to select appropriate thickness and material, it is not practical to perform repeated and expensive mechanical testing of covered stents with coverings of various elastomeric materials and thickness. Therefore, it is advantageous to perform finite element analysis to assist in designing stent coverings and selecting materials for the cover.

### Example 5

### Finite Element (FE) Analysis of Covered Microstents

Currently available neuroendovascular devices are inadequate for effective treatment of many wide-necked, fusiform intracranial aneurysms and intracranial arteriovenous fistulae (AVF). Placing a covered microstent across the intracranial aneurysm neck and AVF rent could restore normal vessel morphology by preventing blood flow into the aneurysm lumen or AVF rent.

Although a substantial amount of numerical analysis about the mechanical properties of the bare stent has been performed, no literature has reported on numerical analysis of covered stents prior to this study. The purpose of this study was to determine the mechanical properties of the covered microstent and investigate the effects of the covering on the mechanical behavior of the covered microstent by using finite element method. Mechanical properties such as deployment pressure, elastic recoil and longitudinal shortening of the covered microstent have been investigated. As described below, contact between the stent and the covering show an impact on the mechanical behavior of the covered stent. The expansion pressure required to inflate the covered stent was directly proportional to the thickness of the covering. The reduced value of both the thickness and Young's modulus of the covering resulted in a reduced expansion pressure and elastic recoil. The covering material affects longitudinal shortening.

In this work, an ultra thin silicone covering of appropriate size was mounted onto a micro Palmaz-Schatz stent before deployment. The effects of the covering on the primary mechanical performance such as deployment pressure, elastic recoil was investigated using finite element (FE) method. The results were compared to the mechanical properties of the microstent without covering. This experiment is described in Gu, L. et al., (Journal of Biomechanics, 2005, 38(20050:1221-1227), which is incorporated herein by reference in its entirety.

Finite element analysis has been used to study mechanical properties of metallic stents, such as deployment pressure, elastic recoil and flexibility. Several earlier studies have addressed effects of stent geometries, the interaction between the stent and catheter balloon, or arterial wall. Auricchio et al. (2000) studied the biomechanical interaction between a balloon-expandable stent and a stenotic artery. Etavea et al. (2001) determined the exact mechanical characteristics of two different types of stents: tubular stents and coil stents. Migliavacca et al. (2002) investigated the effects of different geometrical parameters of a typical diamond-shaped coronary stent on the mechanical performance, and gave some suggestions for optimizing stent shape and performance. Rogers et al. (1997) studied a 2D balloon-artery interaction. However, no finite element analysis of covered stents has been published. This work utilizes the finite element method to predict the mechanical properties of covered microstents.

### Analysis and Modeling

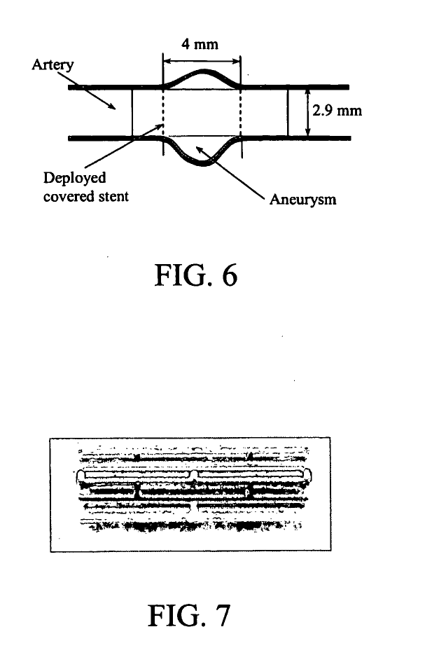

An intracranial artery segment of 2.9 mm lumen diameter with a 4 mm long fusiform aneurysm is depicted in FIG. 6. This lesion will be treated with covered microstent. The covered microstent is placed as shown, across the aneurysm neck, and inflated by the balloon to 3 mm diameter to treat this lesion.

### Geometry and Material Properties

A balloon-expandable Palmaz-Schatz microstent PS154 (Johnson & Johnson, Warren, N.J., USA) was modeled in this study. As shown in FIG. 7, it is a hollow tube with laser cut slots on it. The modeled stent has an outer diameter of 1.47 mm, a length of 8.06 mm, a thickness of 0.1 mm with 2 slots in the longitudinal direction and 12 slots in the circumferential direction. The dimension of each slot is 3.62 mm×0.22 mm. The distal strut length is 0.3 mm, the inner strut length is 0.22 mm, and the metal strut width is 0.14 mm.

The stent is mounted on a balloon microcatheter in its constricted form. The loading and unloading history of the microstent relate to the large plastic deformation. In the absence of detailed material information from the manufacturer, the material properties of the stent were adopted from the published literature (Auricchio et al., 2000; Migliavacca et al., 2002). The microstent was assumed to be made of 316LN stainless steel. The material properties that were used for the analysis are: Young's modulus E=19.6 GPa; Poisson ratio v=0.3; Yield stress σ_{Y}=205 Mpa; Limit stress σ_{M}=515 MPa; Limit nominal strain ε_{M}=60%. The plastic behavior of the microstent was modeled assuming isotropic hardening between yield stress and ultimate stress.

This model considered an ultrathin elastomeric tubular covering captured onto the metal PS154 microstent. In order to address the frequent covering migrations described in the literature (Schellhammer et al., 1999), the present inventors left 0.1 mm of the stent uncovered on each end of the 8.06 mm long microstent. After deployment, the uncovered stent ends will have a subtle trumpet-like flare. The uncovered portion of the stent provides a better grip on the arterial wall to prevent covered microstent migration.

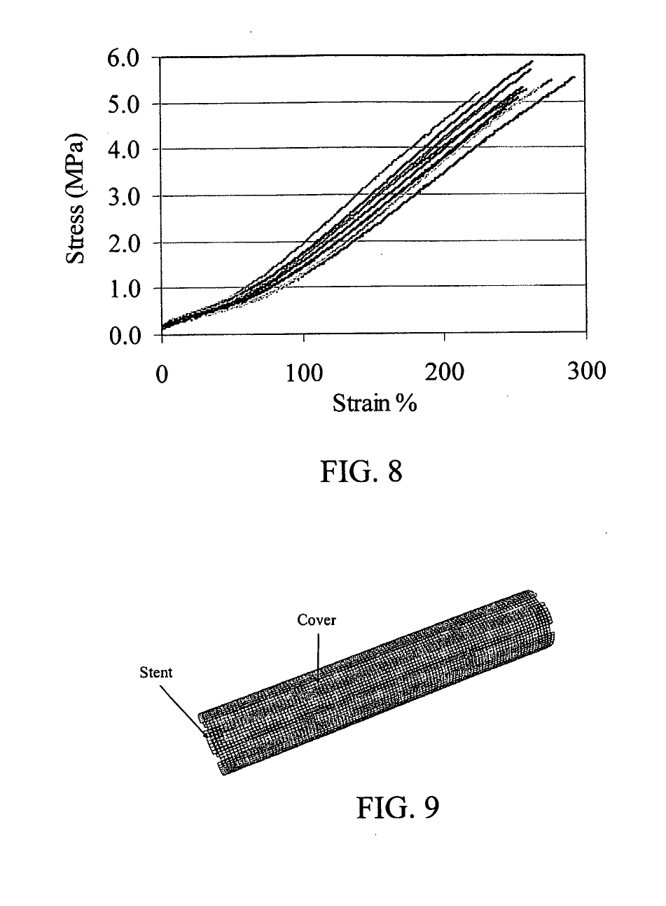

Silicone coverings were made from Silastic® T-2 base and Silastic T-2 curing agent (Dow Corning®, Midland, Mich.). The material properties such as elastic modulus and rupture stress/strain of the silicone covering were measured using an Instron model 4301 testing instrument. Complete stress versus strain profiles for the ten samples was thus obtained, as shown in FIG. 8. The average modulus was 2.47 (±0.14) MPa, and the strain at break is 259 (±19.12)%. In the finite element modeling, it was assumed that the cover has linear mechanical behavior with Young's modulus of 2.47 MPa and Poisson's ratio of 0.3.

### Simulation

FE analysis is widely used as a tool to provide cost-effective information on product design and test as part of the product development process. Considering non-linear large plastic deformation of the metallic stent and the tight contact between the stent and the covering in the present study, the present inventors used commercially available finite element software: ABAQUS 6.3, from Hibbitt, Karlsson & Sorensen, Inc., Rhode Island, USA.

The 3D geometry of the microstent and covering were developed using commercial software I-DEAS 9 (from EDS, Texas, USA) that has the capability to create complex solid and surface models, as well as mesh. The whole stent was meshed with 4-node general shell elements S4R. The stent is discretized into 2730 elements with average length of 0.0796 mm and 3936 nodes. The covering is discretized into 6272 elements and 6336 nodes with the element length of 0.08 mm (FIG. 9).

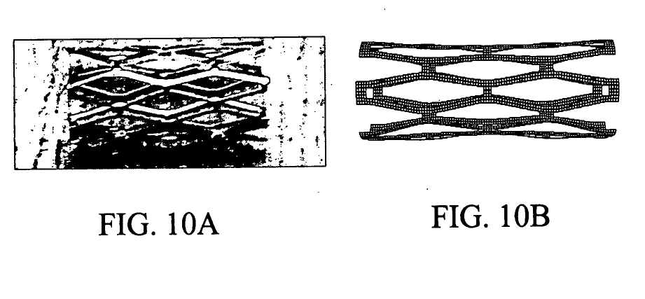

Careful observation of the in vivo stenting experiments in the present inventors' laboratory shows that the balloon is almost uniformly inflated except at two ends, and the stent is expanded by the uniformly inflated part of the balloon. The free ends of the stent are easier to expand than the central part. Thus, the distal ends of the stent expand faster at the beginning of the expansion, then the ends lose contact with the balloon and no pressure from the balloon acts on the ends of the stent. The central portion of the stent continues to be expanded by the balloon until the stent is almost evenly expanded (FIG. 10A). Therefore, in this study, all rotational degrees of freedom were fixed at each end nodes of the stent. Using this model, a uniform pressure was applied on the internal surface of the covered microstent. During loading, pressure was increased until the final diameter of the stent reached the value of 3 mm, and then the pressure was unloaded to study recoil. A non-sliding contact between the covering and the microstent was prescribed. The initial stress of the stent and covering were neglected. The different thickness of 0.08 mm, 0.1 mm, 0.12 mm and 0.15 mm for the covering were tested under the radial pressure load.

### Results and Discussion

A major objective of this work is to compare the mechanical properties of covered microstent and the stent without covering (or bare stent). The stent was expanded by uniform internal pressure which was applied in small increments. The final diameter of the microstent increased to more than twice of its initial diameter. Large structural deformation and material nonlinearities pose considerable difficulties in the analysis. ABAQUS provides an automatic mechanism for stabilizing this kind of problems through the automatic addition of volume-proportional damping to the model. The mechanism is triggered by including the STABILIZE parameter on any nonlinear quasi-static procedure (ABAQUS 6.3-1 documentations, 2002).

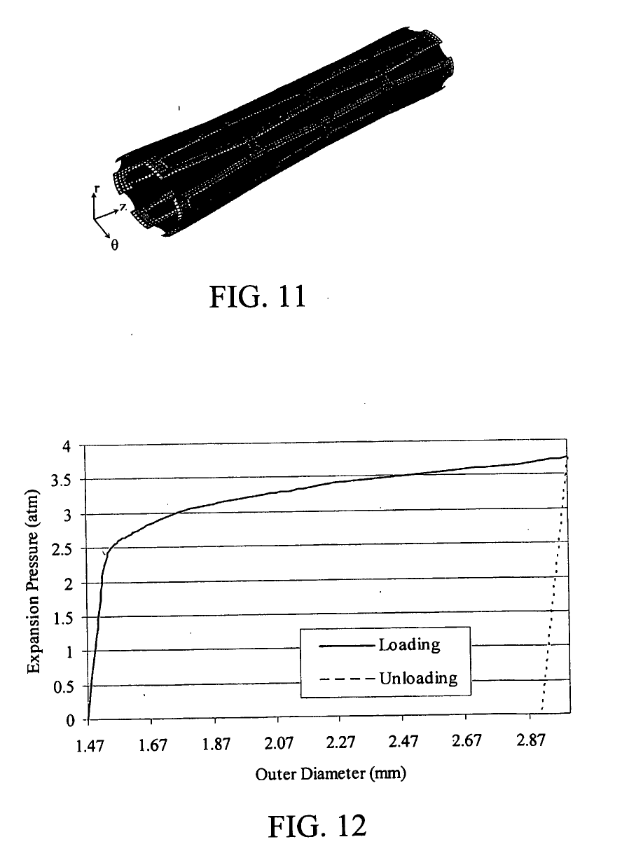

The final state of the bare stent, after expanding from the initial outer diameter of 1.47 mm to 3 mm is shown in FIG. 10B. This is consistent with the experiment (FIG. 5). The expanded stent has a diamond-shaped slot. In the final expansion state, the diameter at the ends of the stent is a little larger than at the center. Furthermore, from finite element analysis results, it can be seen that there is stress concentrate at the joints, as expected. FIG. 11 shows the deformation of the covered stent under the uniform internal pressure. It is clear that the covering expands together with the stent in the areas where they are in contact, indicating that the contact model worked as expected. The uncovered regions at the ends of the stent expanded more easily than the covered part. This prevents the dislocations of the covering. This is consistent with the observations during lab tests.

The relation between the required pressure load and the expanded central diameter of the bare stent is depicted in FIG. 12. It shows that the pressure jumped dramatically from 0 to 2.6 atm mostly during the elastic deformation when the diameter increases by 0.1 mm for the bare stent. The ratio of pressure change over diameter change is 26.2 atm/mm. Then the curve becomes almost flat during the plastic deformation. The pressure increased just 1.18 atm to expand the stent until 3 mm, and the ratio of pressure increase over diameter increase is only 0.82 atm/mm. This was expected based on the stress-strain relation of the stent material. The stent experiences large plastic deformation from 1.57 mm to 3 mm in diameter. The pressure does not increase much during this plastic deformation range because of the low rate of hardening of the stent material. After being unloaded, the stent recoils back along a line parallel to the straight-line portion of the loading curve.

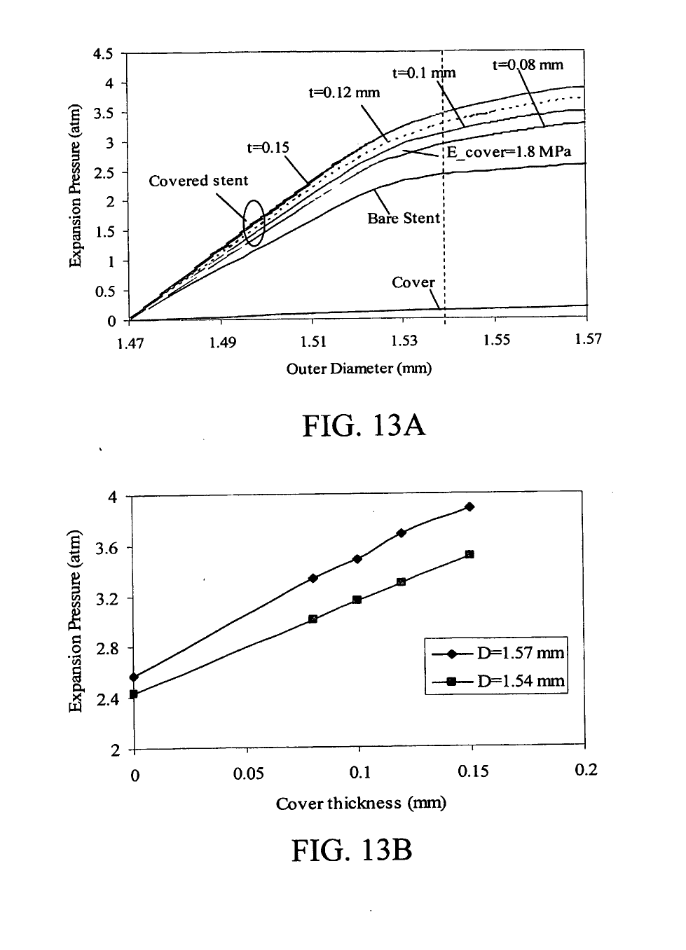

For the covered microstent, the expansion pressure versus deformation plot (FIG. 13A) has the same trend as the plot for bare stent. The pressure jumps during the first 0.1 mm deformation. Beyond 0.1 mm deformation point (D=1.57 mm), the pressure does not increase significantly. The figure also shows pressure versus deformation for many different covering thicknesses and different Young's modulus. As a reference, FIG. 13A includes the pressure versus deformation plot for the bare stent and a 0.1 mm covering when it is expanded without mounting it on a stent.

When a 0.1-mm-thick covering was used, the required pressure increases by approximately 30% compared to the bare stent at a deformation diameter of 1.57 mm. Note that the required pressure to expand covered stent is not equal to the sum of the pressure for bare stent and the covering. This is due to the non-sliding contact between the microstent and the covering. Different covering thicknesses (0.08 mm, 0.1 mm, 0.12 mm and 0.15 mm) were tested. FIG. 13B shows a plot of change in deployment pressure versus thickness of the covering. The two curves were obtained by determining the deployment pressure required for expanding the covered microstent with various covering thicknesses to the diameter of 1.54 and 1.57 mm respectively. The results in the figure predict that the required expansion pressure increases almost linearly with the thickness of the covering. FIG. 13A also shows the influence of material properties. One of the curves corresponds to a covering of 0.1-mm-thickness with a lower Young's Modulus, E_covering=1.8 MPa. Pressure at diameter of 1.57 mm decreased by 4% when the Young's Modulus is decreased from 2.47 MPa to 1.8 MPa. It is clear that a thinner and softer covering is preferred in the design of the covered microstent, considering the expansion pressure.

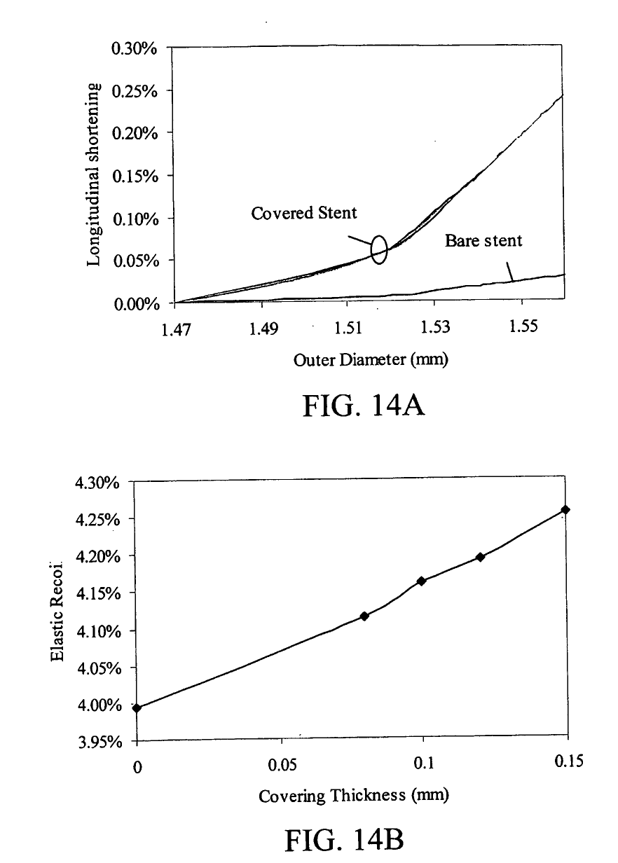

Other factors to be considered in the design of covered microstents include longitudinal shortening and elastic recoil. Longitudinal shortening is the relative difference between the initial length of the stent Lₒ and the expanded length L_{load}. Elastic recoil is defined as the difference between the diameter at expanded state D_{load} and stent diameter after withdrawing the expansion pressure D_{unload}. As shown in FIG. 14A, the longitudinal shortening remained almost unchanged with different covering thicknesses. But there is a large difference between the covered microstents and bare stent. This is because the covering alone has a much larger longitudinal shortening than the bare stent. Therefore when covering is captured onto the stent, it causes a larger longitudinal shortening for the covered stent. The elastic recoil determines the final diameter of the covered stent after the balloon withdraws. As shown in FIG. 14B, the elastic recoil increases almost linearly with the covering thickness.

### Conclusions

3D finite element models have been developed to study the mechanical properties of the covered microstent such as expansion pressure, elastic recoil and longitudinal shortening. The effects of the covering on the mechanical response of the covered microstent were obtained. Results show how the contact between the stent and the covering influences the mechanical behavior of the covered stent. The expansion pressure required to inflate the covered stent was found to be proportional to the thickness of the covering. A thinner covering with low Young's modulus resulted in a reduced expansion pressure and elastic recoil. Longitudinal shortening is affected by the covering material. However, it is also important to ensure that a covered microstent with a thinner cover can be expanded to the desired diameter without rupture of the covering.

This work is important for custom design of the covered microstent such as adding cutout holes to save the perforating arteries. Further modeling will be performed on the design of covering patches, other types of the covered microstent, and different covering materials.

### REFERENCES

ABAQUS 6.3-1 documentations, 2002. Hibbitt, Karlsson & Sorensen, Inc., Rhode Island, USA.
Auricchio, F., Loretob, M. D., Saccoc, E., 2000. Finite-element analysis of a stenotic artery revascularization through a stent insertion. Computer Methods in Biomechanics and Biomedical Engineering 00, 1-15.
Braun, I. S., Hoffman, J. C., Casarella, W. J., Davis., P. C., 1985. Use of coils for transcatheter carotid occlusion. American Journal of Neuroradiol 6, 953-956.
Dandy, W. E., 1938, Intracranial aneurysm of internal carotid artery cured by operation. Annals of Surgery 107, 654.
Debrun, G. 1979. Cerebral aneurysms: advances in diagnosis and therapy. In: Pia, H. W., Langmaid, C., Zierski, J. (Eds.), Special Therapeutic Problems: Springer-Verlag, New York, pp. 228-236.
Dumoulin, C., Cochelin, B., 2000. Mechanical behavior modeling of balloon-expandable stents. Journal of Biomechanics 33, 1461-1470.
Etave, F., Finet, G., Boivin, M., Boyer, J., Rioufol, G., Thollet, G., 2001. Mechanical properties of coronary stents determined by using finite element analysis. Journal of Biomechanics 34, 1065-1075.
Ewald, C., Kühne, D., Hassler, W. E., 2000. Bypass-surgery and coil-embolisation in the treatment of cerebral giant aneurysms. Acta Neurochir 142, 731-738.
Fontaine, A. B., Borsa, J. J., Hoffer, E., Bloch, R., So, C., 2001. Evaluation of silicone as an endovascular stent membrane: In vivo canine studies. Cardiovascular and Interventional radiology. 24(5), 324-328.
Geremia, G., Bakon, M., Brennecke, L., Haklin, M., Silver, B., 1997. Experimental arteriovenous fistulas: treatment with silicone-covered metallic stents. American Journal of Neuroradiology 18(2), 271-277.
Giannotta, S. L., Litofsky, N. S., 1995. Reoperative management of intracranial aneurysms. Journal of Neurosurg 83, 387-393.
Johnston, S. C., Zhao, S., Dudley, R. A., Berman, M. F., Gress, D. R., 2001. Treatment of unruptured cerebral aneurysms in California. Stroke 32, 597-605.
Laitinen, L., Servo, A., 1978. Embolization of cerebral vessels with inflatable and detachable balloons—technical note. Journal of Neurosurg 48, 307-308.
Marty, B., Leu, A. J., Mucciolo, A., von Segesser, L. K., 2002. Biologic fixation of polyester- versus polyurethane-covered stents in a porcine model. Journal of Vascular and Interventional Radiology 13(6), 601-607.
Migliavacca, F., Petrini, L., Colombo, M., Auricchio, F., Pietrabissa, R., 2002. Mechanical behavior of coronary stents investigated through the finite element method. Journal of Biomechanics 35, 803-811.
Najibi, S., Bush, R. L., Terramani, T. T., Chaikof, E. L., Gunnoud, A. B., Lumsden, A. B., Weiss, V. J., 2002. Covered stent exclusion of dialysis access pseudoaneurysms. Journal of Surg Res. 106(1), 15-19.
Rogers, C., Tseng, D. Y., Squire, J. C., Edelman, E. R., 1997. Balloon-artery interactions during stent placement: a finite element analysis approach to pressure, compliance, and stent circulation. In Proceedings of the 23rd Annual Meeting of the Society for Biomaterials. New Orleans, La.
Schellhammer, F., Wahkloo, A. K., Nagursky, H., Schumacher, M., 1999. Vein graft-coated stents for endovascular occlusion of canine experimental arteriovenous fistulae. Invest. Radiol. 34(1), 22-27.

### Example 6

### Polymer Coverings for Microstent-Grafts in Neurosurgery

Ultrathin, non-porous polymer coverings for microstent-grafts were developed for the endovascular treatment of intracranial aneurysms, particularly wide-necked aneurysms, and intracranial arteriovenous fistulae. It was hypothesized that introducing these coverings will prevent the blood flow into the aneurysmal sac and eliminate the possibility of aneurysm rupture. These coverings were made from exceptionally tough, biocompatible, elastomeric, and low-cost materials using a conventional polymer processing technique. A balloon expandable microcatheter carried the polymer-covered microstent at the distal end for delivery. When deployed, polymer coverings remained secured on the stent and stretched uniformly along with it because of the unique elastomeric properties of the covering. Covered microstent recoil due to the elastomeric radial force was measured at each deployment pressure and compared with the bare metallic microstent deployed at the same pressure. A three-point bend test was also performed to assess flexibility of each covered stent, compared to each other and compared to the naked control stents. The in vitro results showed that all these coverings had very low impact on recoiling and flexibility, which confirms their suitability as stent coverings. In this analysis, the present inventors describe materials processing, manufacturing of these coverings, and their mechanical in vitro testing.

### Materials and Methods

Tegaderm™ Stent Covering

Tegaderm™ adhesive transparent dressing (3M, city, state) was used as a microstent covering by wrapping it around the metal stent. The 40 μm thick tape material is a flexible polyurethane with adhesive on one side. In this experiment, 1×, 2×, and 3× wrapping of Tegaderm™ tape were studied. Tegaderm™ is a simple, inexpensive method to cover microstents on the sterile endovascular table just prior to stent deployment. The advantages of Tegaderm™ as a stent covering is its ultra-thin, flexible design, simplicity, inexpensiveness, availability, and minimal preparation. The disadvantages of Tegaderm™ are tissue reaction to the exposed adhesive, variability at placement, and the obligatory seam where the ends of the adhesive meet on the stent.

Silicone Stent Coverings

Silicone stent coverings were made from Silastic® T-2 base and Silastic T-2 curing agent (Dow Coming®, Midland, Mich.). This is a two component, room temperature vulcanization (RTV), platinum cured silicone elastomer. A mixture of 10 parts of base and 1 part of curing agent was first mixed thoroughly and degassed. About 0.20 mg mixture was applied around a 19 gauge (1.07 mm diameter) syringe needle in such a way that it covered an approximate 18 mm portion along the length of the needle. The needle was then rotated horizontally at 10 rpm by a motor to smear the silicone uniformly. After 24 hours of curing, the silicone stent covering was removed from the needle by a swelling technique. The swelling was performed by submerging the entire silicone stent covering and needle in n-hexane. After the silicone stent covering swelled enough to easily remove it from the needle, the n-hexane was evaporated, either under room-air temperature or under vacuum, which caused the stent covering to return to its original size of 1.07 mm diameter. Under the microscope, a 1.5 mm portion of the covering was cut from both ends with a sharp razor blade in order to eliminate any non-uniformity, which can occur at the terminal ends. For the present experiment, a covering length of 14.5 mm was utilized to mount on a 17.5 mm length stent. This allows a 1.5 mm portion of the stent to remain naked at both ends, which should maintain the stent's metallic grip to the vessel inner wall. Using this procedure, a batch of 15 to 20 coverings were made at a time. These coverings are uniform, seamless, and smooth. Before mounting onto the microstent, each covering was cut the appropriate length and inspected under the microscope. The average covering thickness measured optically under a microscope was 200 μm. Any commercially available microstent can be covered with this technique. The length and diameter of the stent covering can be modified easily to custom fit the chosen microstent. The most widely available stents for intracranial use at this time are balloon-inflatable coronary microstents such as the S6760 or 58 (AVE, Medtronic, city, state), or the Bx Velocity™ (Cordis, Miami Lakes, Fla.).

Polyurethane Coverings

Tecoflex® SG-80A, a polyether-based thermoplastic polyurethane of solution processible grade, was obtained from Thermedics Polymer Products (Woburn, Mass.) in the form of beads. 10 wt %// Tecoflex® solution in N,N-dimethylacetamide (DMAC) was made using vacuum dried beads. Uniform polymer coating was applied first on 19 gauge (1.07 mm diameter) syringe needles by dip-coating method. Dip-coating 7 times, dipping at a withdrawal rate of 4 mm/sec and waiting at a 3 minute interval between each 2 successive dipping maneuvers formed a stent covering 90 μm thick. Coated needles were then heat treated at 55° C. for 3-4 hours under horizontal rotation (10 rpm) in an isotemp incubator (Robbins Scientific, city, state, Model 400) followed by freeze drying (Labconco, city, state Freeze Dryer 4.5) for overnight at −40° C. and 30 μm of mercury pressure. This step was necessary to get DMAC-free, uniform coatings. The polymer coating was then removed as thin walled tubings from the needles using a swelling technique. Seventy percent ethanol in water was used as the swelling solvent for the polyurethane. The stent covering swelled by 20% in about 20 minutes when completely submerged in the 70% ethanol. The polyurethane stent covering was then removed from the needle and dried at either room air temperature or vacuum. After drying, the polyurethane stent covering returned to its original size and shape (1.07 mm diameter). Because of the higher tear-strength of polyurethane compared to silicone, we were able to make the polyurethane covering thinner—90 mm versus 200 mm.

Coated Silicone Rods For In Vitro Testing of the Various Stent Coverings

1.1 mm diameter silicone rods were made from Silastic® T2 (Dow Corning®, Midland, Mich.) silicone elastomer, which is a two component RTV, platinum cured silicone. Ten parts of the Silastic® base and one part of the Silastic® curing agent (wt/wt) were mixed thoroughly and degassed under a vacuum to remove trapped air bubbles. 1.12 mm ID, 100 mm long Kimax-51 glass capillary tubes (Kimble-Kontes, city, N.J.) were used as molds for making silicone rods. Glass capillaries were filled first with Silastic® mixture by vacuum suction, and then both ends were sealed with paraffin wax. After 24 hour curing, capillaries were immersed in a 48% hydrofluoric (HF) acid bath (Sigma-Aldrich Corp., St. Louis, Mo.). Glass capillaries were dissolved completely in approximately 20 minutes. Silicone rods were then collected after repeated washing in deionized (DI) water and acetone followed by vacuum drying. Because the Tegaderm™ will not adhere to the silicone surface, a co-polymer coating of approximately 10 mm in thickness was applied to the silicone surface. Dry silicone rods were then dip-coated with 10 wt % CarboSil 40 90A, a silicone-polyurethane copolymer (The Polymer Technology Group Inc., Berkeley, Calif.) solution in THF (define). The thickness of the coating was approximately 10 μm.

Radial Force Measurement

The radial force due to recoiling of the covered microstents was measured with respect to the deployment pressure and compared with the bare metallic microstent.

Mechanical Testing (3-Point Bend Test)

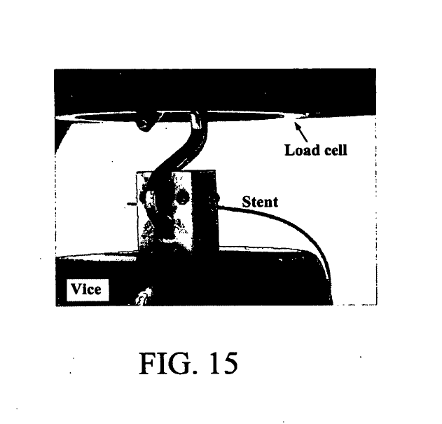

3-point bend tests were performed to determine the mechanical properties, in particular the stiffness, of stent coverings. Bend tests were performed using an Instron model 4301 testing instrument and an Instron #2511-101 load cell (Canton, Mass.). A 100 g Ohaus weight (Pinebrook, N.J.) was used to calibrate the load cell prior to testing. Data was collected using Instron Series IX software version 8.07. For each test, an S shaped hook, attached to the load cell, pulled the stent upward against two fixed cylindrical supports. The cylindrical supports were housed in a fixture, which was held by a vise below the Instron cross-head. The distance between supports is 12.1 mm and the diameter of supports is 3.05 mm. FIGS. 1A-1C are diagrams showing the S hook (FIG. 1A) and fixture (FIGS. 1B and 1C). FIG. 15 is a photograph of the actual apparatus including the S hook, stent and 3-point bend test fixture.

In Vitro Covered Microstent Testing

Due to the expense and limited availability of stents, only one sample of covered microstent was tested in vitro for each covering material. However, each covered stent was tested 8 times to determine the repeatability of the testing technique. The maximum deflection was 1 mm, since the soft, stainless steel stents undergo considerable permanent deformation when deflected past 1 mm. Some permanent deformation occurred between tests; therefore, stents were manually straightened between tests.

In Vitro Testing of Covered Silicone Rods

Covered silicone rods were prepared with various covering materials by placing them around the coated silicone rods. The purpose was to provide a substrate for bend testing of the coverings that was more compliant than the stent. The more compliant substrate allowed for easier comparison of coverings since a larger proportion of the measured load is due to the coverings rather than the substrate. The silicone rods have the additional advantage of being easier and far less expensive to produce than the stents. Both silicone and Tecoflex® tubing were swelled in appropriate solvent first and then placed over the substrate. Tegaderm™ adhesive was cut in right sizes before they were wrapped around the substrate. Four covered silicone rods were tested for each covering material.

### Results and Discussion

Covered Stents

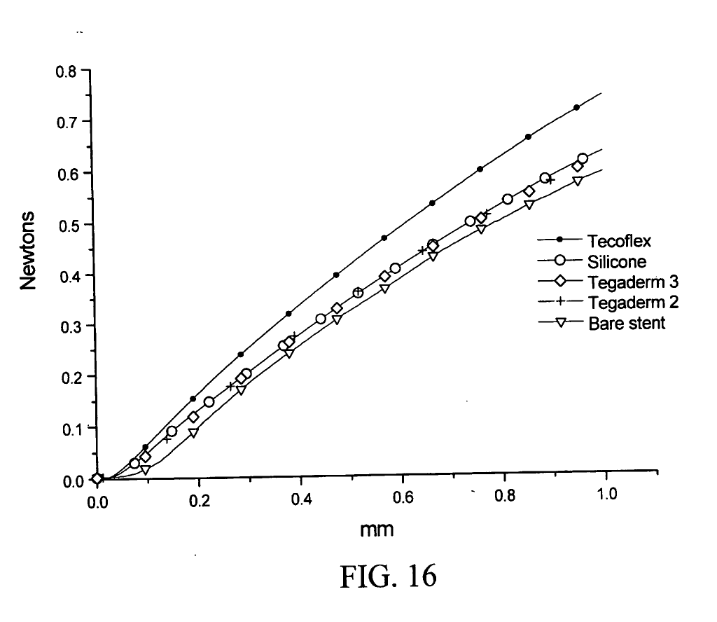

Since stents are fed through the tortuous vasculature, one obvious functional requirement of stents is flexibility. Since the stent has been modified with various coverings, 3-point bend testing was performed to determine if the stents remained flexible following coating. FIG. 16 shows the force (newtons) verses displacement (mm) graphs for the covered stents compared with the bare stents. The displacement represents the displacement of the center of the stent as it is pulled by the S-shaped hook. Each curve is the average of the 8 tests for each stent. The standard errors for the tests show that the bend test is highly reproducible. Table 2 shows a summary of the data for force measured at the maximum displacement for each test. This is the maximum force for each test and is indicative of the stiffness of each stent. The Tecoflex® covered stent was measured to be 25% stiffer than the bare stent; while the other covered stents indicated an increase in stiffness of less than 10%. Of course, since only one sample was tested for each covering, it is unclear whether the differences in stiffness measured are due to the covering material or due to variations in the stents themselves. Finally, since the largest difference measured is 25%, it appears that none of the coverings have a mechanically significant effect on the stiffness of the stent.

TABLE 2
Maximum force measured at 1 mm displacement for bare and covered
stents. 1000 mN = 1 newton.

| | | Std. | Std. | % Increase in |
|---|---|---|---|---|
| | Max force (mN) | Deviation | Error | force compared to |
| Group | avg. of 8 tests | (mN) | (mN) | bare |
| TECOFLEX | 737 | 20 | 7 | 25 |
| Silicone | 629 | 11 | 4 | 7 |
| TEGADERM 3 | 620 | 7 | 3 | 5 |
| TEGADERM 2 | 617 | 18 | 6 | 5 |
| Bare | 590 | 18 | 6 | — |

TABLE 3
Summary of mechanical testing data for covered silicone rods. Values
are average of 4 samples. Standard error is in parenthesis. Load carried by cover
estimated by subtracting measured average load carried by bare rod.

| | | Force (mNewtons) | | | |
|---|---|---|---|---|---|
| | | 1 mm deflection | | 3 mm deflection | |
| | samples | Cover + rod | Cover (est.) | Cover + rod | cover (est.) |
| Bare rod | 4 | 6.9 (0.7) | — | 17.6 (1.9) | — |
| Silicone | 4 | 19.4 (1.9) | 12.5 | 63.2 (2.9) | 45.6 |
| TECOFLEX | 4 | 19.2 (1.4) | 12.4 | 65.0 (5.2) | 47.4 |
| TEGADERM 1X | 4 | 17.1 (1.0) | 10.2 | 46.5 (6.1) | 28.9 |
| TEGADERM 2X | 4 | 27.5 (1.0) | 20.7 | 75.0 (3.6) | 57.5 |
| TEGADERM 3X | 4 | 43.2 (1.5) | 36.4 | 126.0 (4.1) | 108.4 |

TABLE 4
Results of Tukey's multiple comparison test for load at 1 and 3 mm.
Testing performed on covered silicone rods.

Statistical
Difference
(p < 0.05)

| Covered silicone rod sample | 1 mm | 3 mm |
|---|---|---|
| TEGADERM-3X vs. BARE | Yes | Yes |
| TEGADERM-3X vs. TEGADERM-1X | Yes | Yes |
| TEGADERM-3X vs. SILICONE | Yes | Yes |
| TEGADERM-3X vs. TECOFLEX | Yes | Yes |
| TEGADERM-3X vs. TEGADERM-2X | Yes | Yes |
| TEGADERM-2X vs. BARE | Yes | Yes |
| TEGADERM-2X vs. TEGADERM-1X | Yes | Yes |
| TEGADERM-2X vs. SILICONE | Yes | No |
| TEGADERM-2X vs. TECOFLEX | Yes | No |
| TECOFLEX vs. BARE | Yes | Yes |
| TECOFLEX vs. TEGADERM-1X | No | No |
| TECOFLEX vs. SILICONE | No | No |
| SILICONE vs. BARE | Yes | Yes |
| SILICONE vs. TEGADERM-1X | No | No |
| TEGADERM-1X vs. BARE | Yes | Yes |

Covered Silicone Rods

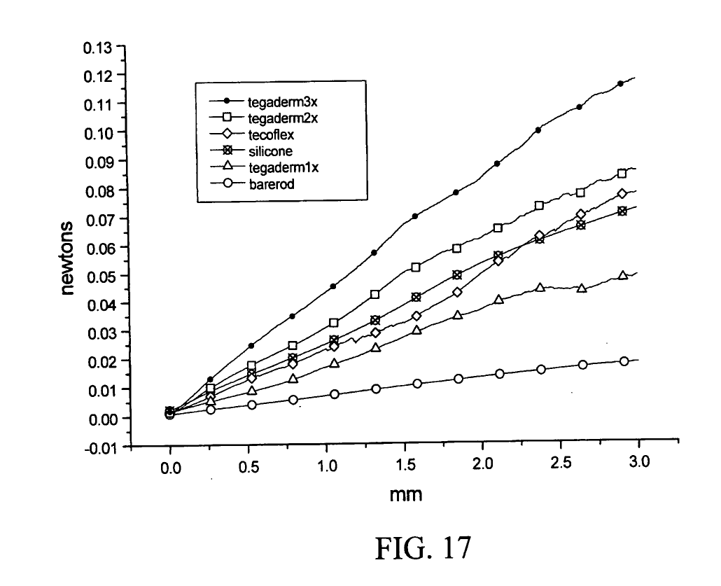

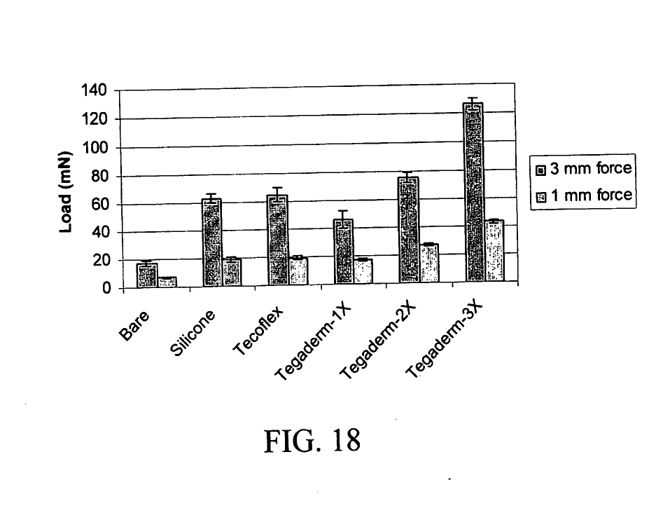

Due to their low stiffness, high elasticity and low cost, silicone rods were used as a substrate for bend testing of the coverings. FIG. 17 shows the load vs. displacement curves for each covering material and the bare silicone rod. Each curve is one representative sample for each covering material. The loads vs. displacement curves are reasonably linear, which is expected since all covering materials are elastomers. FIG. 18 summarizes the bend test data by showing the force measured for 1 and 3 mm displacements. All covering materials increased the stiffness of the silicone rod by a large amount. This was a desirable result since the differences between covering materials are more easily recognized for a more compliant substrate. The Tegaderm™ 3× is considerably stiffer than the other samples, while it appears that the remaining samples are comparable. Table 3 shows the exact values for load at 1 and 3 mm that were represented in the bar graph form of FIG. 18. Table 3 also shows estimates for the load carried by the covering, which was determined by simply subtracting the average load for bare rods for 1 and 3 mm of deflection.

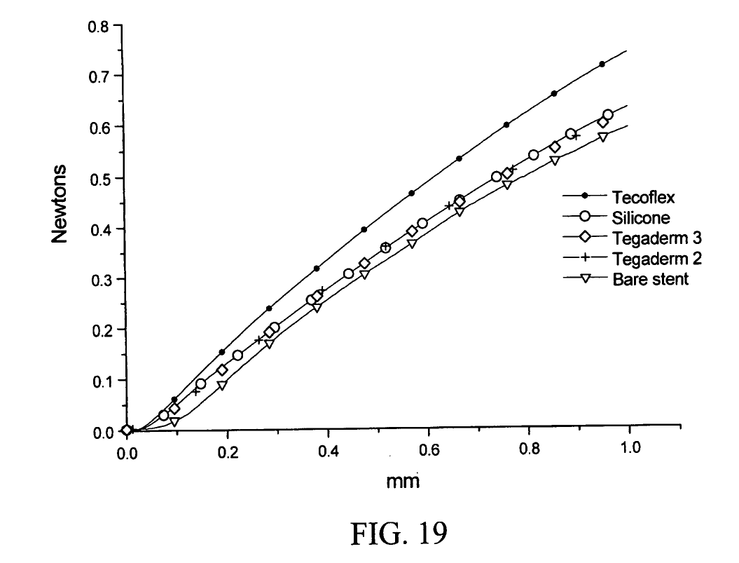

FIG. 19 shows load vs. displacement curves for the bare stent compared to the coverings. Since the slope of the metal stent curve is much steeper than for the coverings, it is clear that the coverings do not increase the stiffness of the stent in a mechanically relevant manner. The small stiffness of the coverings compared to the stents is also apparent when comparing Table 2 and Table 3. The load at 1 mm displacement for the metal stent is 590 mNewtons (Table 2), while the load estimated for the stiffest covering, Tegaderm™ 3×, is 43 mNewtons (Table 3). The stiffest covering is therefore is less than 10% of the stiffness of the bare stent.

Even though the coverings are very compliant compared to the metal stents, the variations in stiffness between coverings are of interest if they are to be used for other applications. Therefore, 1-way ANOVA analysis was used to determine if the different coverings showed statistically different values for stiffness. Separate ANOVAs were performed on load vs. displacement data for 1 and 3 mm deflection. For both deflections, ANOVA indicated a statistical difference among the 6 sample groups with p<0.001. Tukey's multiple comparison test with p<0.05 was used to identify the locations of the differences. Table 4 shows the locations of the differences. For both 1 and 3 mm deflections all samples are significantly different from the bare rod, which is not surprising since the measured stiffnesses (loads) were at least 3 times higher for all covered rods compared to bare. For both 1 and 3 mm deflection, the measured loads for Tegaderm™ 3× are statistically different than all samples. Looking back to FIG. 18, Tegaderm™ 3× is much stiffer than the other coverings. Similarly, FIG. 18 and Table 4 indicate that Tegaderm™ becomes significantly stiffer as it is wrapped from 1 to 2 to 3 times. Of course, this is not surprising since the thickness of the Tegaderm™ is increased as it is wrapped. Finally, a statistical difference was found for Tegaderm™ 2× between both silicone and Tecoflex® at 1 mm, but not for 3 mm. This can be accounted for by the fact that the mean loads for Tecoflex®, silicone, and Tegaderm™ 2× are fairly close in value. If the mechanical testing were repeated again, the multiple comparison procedure would probably yield different results for Tegaderm™ 2× compared to silicone and Tecoflex®.

As the stent is deployed, the covering of the present invention stretches uniformly along with the metallic stent due to its unique elastomeric property. There are several advantages provided by using elastomeric stent coverings of the present invention over the most commonly used polytetrafluoroethylene (PTFE) stent coverings. PTFE coverings are generally fixed with the metallic stent either by fine sewing or by placing it in between two metal stents in a sandwich fashion so that they remain secured. In contrast to the PTFE coverings, the coverings of the present invention are firmly held over the metal stent due to their elastomeric property. Unlike PTFE, the coverings of the present invention can be conveniently placed over the metallic stent by the swelling and shrinking mechanism of the polymer. Also, the ultrathin elastomeric coverings of the present invention can be easily carried by a microstent to the intracranial site of the brain.

It should be understood that the examples and embodiments described herein are for illustrative purposes only and that various modifications or changes in light thereof will be suggested to persons skilled in the art and are to be included within the spirit and purview of this application.

1. A flexible stent covering comprising a polymer tube having a thickness within the range of 20 microns and 200 microns.

2. The stent covering of claim 1, wherein said stent covering has a thickness of 150 microns.

3. The stent covering of claim 1, wherein said polymer tube comprises an elastomer.

4. The stent covering of claim 1, wherein said polymer tube comprises a thermoplastic elastomer.

5. The stent covering of claim 1, wherein said polymer tube comprises a thermoplastic elastomer selected from the group consisting of polyurethane, silicone-polyurethane copolymer, styrene-ethylene-butylene-styrene, and silicone.

6. The stent covering of claim 1, wherein said polymer tube is lubricated.

7. The stent covering of claim 1, wherein said polymer tube is seamless.

8. The stent covering of claim 1, wherein said stent covering is swellable.

9. The stent covering of claim 1, wherein said stent covering occupies a rolled configuration.

10. The stent covering of claim 1, wherein said stent covering is impregnated, or coated with, one or more biologically active agents.

11. The stent covering of claim 10, wherein said one or more biologically active agents comprise one or more substances that degrade vascular occlusions or prevents the formation of vascular occlusions.

12. The stent covering of claim 10, wherein said one or more biologically active agents are released from said stent covering in a controlled release fashion.

13. The stent covering of claim 10, wherein said one or more biologically active agents are released or become activated upon exposure to blood.

14. The stent covering of claim 10, wherein said one or more biologically active agents are released or become activated at a predetermined pH.

15. The stent covering of claim 1, wherein said stent covering further comprises an imageable material.

16. The stent covering of claim 15, wherein said imageable material is radio-opaque.

17. The stent covering of claim 15, wherein said imageable material comprises gold particles.

18. The stent covering of claim 15, wherein said imageable material comprises at least one material selected from the group consisting of paramagnetic iron oxide, dysprosium (Dys) oxide, and gadolinium (Gd) oxide.

19. The stent covering of claim 15, wherein said imageable material comprises a quantum dot.

20. The stent covering of claim 1, wherein said stent covering has one or more perforations.

21. A covered stent comprising a stent and a stent covering, wherein said stent comprises a hollow cylinder having a lumen, an inner surface defining said lumen, an outer surface, wherein said stent covering covers at least a portion of said stent outer surface, and wherein said stent covering comprises a polymer tube having a thickness within the range of 20 microns and 200 microns.

22. The covered stent of claim 21, wherein said stent is a flexible stent, and wherein said stent covering does not reduce the flexibility of said flexible stent.

23. The covered stent of claim 21, wherein said stent is a metallic stent.

24. The covered stent of claim 21, wherein said stent is a braided or mesh metallic stent.

25. The covered stent of claim 21, wherein said stent comprises a polymer.

26. The covered stent of claim 21, wherein said stent is a radially-expandable stent.

27. The covered stent of claim 21, wherein said stent is a balloon-expandable stent.

28. The covered stent of claim 21, wherein said stent is a self-expanding stent.

29. A method for reinforcing a biological lumen, comprising introducing a covered stent at a target site within the biological lumen, wherein the covered stent comprises a stent and a stent covering and wherein the stent covering comprises a polymer tube having a thickness within the range of 20 microns and 200 microns.

30. The method of claim 29, wherein the biological lumen is selected from the group consisting of a ureter, a urethra, a fallopian tube, a bile duct, and an intestine.

31. The method of claim 29, wherein the biological lumen is a blood vessel.

32. The method of claim 29, wherein the biological lumen is within a human.

33. The method of claim 29, wherein the biological lumen is an intracranial blood vessel within a human.

34. A method for making a covered stent, comprising swelling a polymer tube in a swelling agent and mounting the swelled polymer tube over a stent, wherein the swelled polymer tube has a thickness within the range of 20 microns and 200 microns.

35. The method of claim 34, wherein the swelling agent comprises a solvent.

36. The method of claim 34, wherein the solvent comprises acetone or n-hexane.

37. The method of claim 34, wherein the solvent is a mixed solvent.

38. The method of claim 37, wherein the mixed solvent comprises acetone and water.
